(19) United States
(12) Patent Application Publication
(10) Pub. No.: US 2026/0334869 A1
(43) Pub. Date: Oct. 29, 2026

(54) BIOMARKER IDENTIFICATION AND TREATMENT RECOMMENDATION

(71) Applicant: Bios Health Ltd., Cambridge, Cambridgeshire (GB)

(72) Inventors: Oliver ARMITAGE, Cambridge, Cambridgeshire (GB); Emil HEWAGE, Cambridge, Cambridgeshire (GB); Luca ANNECCHINO, Cambridge, Cambridgeshire (GB); Mike REES, Cambridge, Cambridgeshire (GB); Aleksi TUKIAINEN, Cambridge, Cambridgeshire (GB); Elvijs SARKANS, Cambridge, Cambridgeshire (GB); Antonin BERTHON, Cambridge, Cambridgeshire (GB); Matjaz JAKOPEC, Cambridge, Cambridgeshire (GB); Bernard MAK, Cambridge, Cambridgeshire (GB); Myrta STOUKIDI, Cambridge, Cambridgeshire (GB); Pascal FORTIER-POISSON, Cambridge, Cambridgeshire (GB); Olivier TESSIER LARIVIERE, Cambridge, Cambridgeshire (GB); Sudhakaran SHUNMUGAM, Cambridge, Cambridgeshire (GB); Benjamin APPLETON, Cambridge, Cambridgeshire (GB); Lorenz WERNISCH, Cambridge, Cambridgeshire (GB)

(21) Appl. No.: 19/118,295

(22) PCT Filed: Oct. 3, 2023
(86) PCT No.: PCT/GB2023/052553

§ 371 Date: Apr. 3, 2025

(30) Foreign Application Priority Data
Oct. 3, 2022 (GB) 2214547.8

(51) Int. Cl. A61B 5/00; A61B 5/388

(52) U.S. Cl. CPC A61B 5/4833; A61B 5/388; A61B 5/4842; A61B 5/686; A61B 5/6877

(57) ABSTRACT

The present invention is a clinical recommendation system that comprises one or more modules configured to obtain a set of neural signal data collected from the patient suffering from the illness. The system collects to set of neural signal data with respect to a neural biomarker. The system obtains an assessment based on the set of neural signal data in relation to a response of a neural biomarker from the patient, where the biomarker response is indicative of one or more determinations made with respect to the neural biomarker by the system. The system provides the assessment as a recommendation that corresponds to the patient.

The present application relates to a system, apparatus and method(s) for utilising neural biomarker response in clinical decision-making.

### BACKGROUND

Medical treatments, including drugs, devices, and clinical interventions, are selected for specific patients, or the doses/settings of the treatments are modified and personalised, on the basis of direct observations by the clinician and the reading of clinical biomarkers. These could be from sensors reading physiological signals such as ECG or Blood pressure, from samples collected from the patient such as blood or tissue samples analysed in the lab, or from patient-reported outcome measures like questionnaires.

Presently, the information recorded from the nervous system is rarely included in the current use case for modifying treatments. Rare examples of when it is used are often when the disease is directly neural in nature, such as epilepsy where EEG or ECOG is used in the brain to measure brain waves or use of fMRI to measure brain activity in brain disorders.

It is desired to find a way to utilise information recorded from the nervous system, in particular, to deliver a clinical recommendation with respect to the illness or disease based on one or more responses measured from an identified neural biomarker, potentially in real-time, that would at least address the above technological void.

The embodiments described below are not limited to implementations which solve any or all of the disadvantages of the known approaches described above.

### SUMMARY

This Summary is provided to introduce a selection of concepts in a simplified form that are further described below in the Detailed Description. This Summary is not intended to identify key features or essential features of the claimed subject matter, nor is it intended to be used to determine the scope of the claimed subject matter; variants and alternative features which facilitate the working of the invention and/or serve to achieve a substantially similar technical effect should be considered as falling into the scope of the invention disclosed herein.

Present disclosure provides a system, apparatus, and method(s) for utilising the neural signal data comprising neural information recorded from nerves. The neural signal data are recorded from nerves all over the body and incorporated into the clinical decision-making about treatment selection, dosing, or optimization. An exemplary system would pick a therapy applied to the body on the basis of a reading of the neural signal data.

The neural signals (data) may be recorded from a nerve where bioelectronic therapy is being applied. The resulting neural biomarkers can be used to dose and set up the bioelectronic therapy. Thereby benefits include but are not limited to providing 1) Responder identification; 2) Confirmation of correct device placement; 3) Personalised therapy settings; 4) Personalised avoidance of side effects; 5) Faster time to therapeutic effect; 6) Ability to predict response in diseases with long time scale to conventional endpoints (e.g., Rheumatoid Arthritis).

The neural signals may also be recorded from a nerve that is activated by a drug that is being prescribed. The resulting neural biomarkers can be used to see the effect of the drug in real-time, and can be used to predict which patients are going to respond to this drug. Thereby benefits include but are not limited to providing 1) Key insights for new and existing neurally-mediated assets; 2) Label expansion to other neurally-mediated diseases; 3) Personalised single drug and combination dosing; 4) Responder identification; and 5) Trial stratification.

In a first aspect, the present disclosure provides a clinical recommendation system, wherein the system comprising: one or more modules configured to: obtain a set of neural signal data collected from the patient suffering from the illness, wherein the set of neural signal data is collected with respect to a neural biomarker; obtain an assessment based on the set of neural signal data in relation to a response of a neural biomarker from the patient, wherein the biomarker response is indicative of one or more determinations made with respect to the neural biomarker by the system; and provide the assessment, at least in part, as a recommendation that corresponds to the patient. The system may be configured to provide a recommendation concerning one or more treatments for an illness based on the neural biomarker response.

Preferably, at least one module is configured to: receive the set of neural signal data; determine a baseline for the neural biomarker response from the set of neural signal data in the absence of the treatment; administer a treatment based on a schedule to the patient while obtaining the set of neural signal data collected from the patient; identify one or more changes in the set of neural signal data in response to and for a duration of the treatment; determine the neural biomarker response based on said one or more changes in the set of neural signal data through the time period appropriate to the treatment; ascertain the neural biomarker response in relation to the baseline; provide an assessment of the treatment based on the neural biomarker response; and output the assessment as the recommendation.

Preferably, at least one module is configured to: receive the set of neural signal data; determine a level of the neural biomarker present with respect to a historical patient population with the illness and/or patient specific levels of the neural biomarker associated with the illness; determine a state or severity of the illness based on one or more determined levels of the neural biomarker as compared to the historical patient population or the patient specific levels of neural biomarker characteristics associated with the illness; provide an assessment of one or more treatments based on said determinations, wherein the assessment correlates one or more neural biomarker levels in relation to those of, or known for, the illness; and output the assessment as the recommendation.

Preferably, at least one module is configured to: receive the set of neural signal data; determine, in the absence of treatment for the illness, the neural biomarker response for an expected response of a treatment based on a patient population with the treatment and/or historical levels of the neural biomarker response from a different patient in response to the treatment; provide an assessment of one or more treatments based on the determination in relation to the neural biomarker response historically known for the illness; and output the assessment as the recommendation.

In a second aspect, the present disclosure provides a method (or a computer-implemented method) for providing an assessment based on a neural biomarker response, the method comprising: obtaining a set of neural signal data collected from the patient suffering from the illness, wherein the set of neural signal data is collected with respect to a neural biomarker; obtaining an assessment based on the set of neural signal data in relation to a response of a neural biomarker from the patient, wherein the biomarker response is indicative of one or more determinations made with respect to the neural biomarker by the system; and providing the assessment for the patient

In a third aspect, the present disclosure provides a device for implementing the system according to the first aspect and options described in the following section, wherein the device comprising: at least one component configured to: identify a neural biomarker for use with the system based on the method of claims to the second aspect and options.

In a fourth aspect, the present disclosure provides a system for optimizing a treatment based on a neural biomarker, wherein the system is configured to: perform one or more steps to optimize the treatment or the treatment plan toward the target therapeutic objective for the illness in relation to or as part of the recommendation provided by the system according to the first aspect and options.

In a fifth aspect, the present disclosure provides a method (or computer-implemented method) for applying neural biomarker response in clinical diagnosis and treatment screening, the method comprising: obtaining a neural biomarker response in response to a treatment for an illness exhibited by a patient; determining whether the neural biomarker response meets one or more pre-determined criteria determined based on a patient cohort exhibiting neural biomarker response following successful treatment(s) or recovery of the illness; and providing the treatment based on whether neural biomarker response meets said one or more pre-determining criteria, wherein the treatment is used in relation to the clinical diagnosis and treatment screening.

In a sixth aspect, the present disclosure provides a system for optimizing a bioelectronic treatment for an illness, the system comprising one or more modules configured to: apply an electrical stimulation to a nerve of the patient based on a set of electrical stimulation parameters; obtain a set of neural signal data from the patient in relation to the electrical stimulation; determine one or more neural biomarker responses from changes in: the set of neural signal data indicative of changes in neural activity, wherein the changes are associated with the progression of the illness, in response to the bioelectronic treatment, or when undergoing the bioelectronic treatment, or for a period following the bioelectronic treatment, or a combination thereof; obtain one or more physiological signals of the patient in response to the bioelectronic treatment; and transmit said one or more neural biomarker responses and/or one or more physiological signals to be analysed by an optimizer, wherein the optimizer is configured to: receive said one or more neural biomarker responses and/or said one or more physiological signals as input data to a model or search algorithm related to the model; evaluate the input data based an objective; determine a query point based on the objective; and output the query point; updated the set of electrical stimulation parameters based on one or more outputs from the optimizer.

In a seventh aspect, the present disclosure provides a system for recommending a pharmacological treatment based on a neural biomarker, the system comprising one or more modules configured to: receive a set of neural signal data of the patient; determine a neural biomarker response from one or more changes in the set of neural signal data indicative of neural activity before, during, and after the treatment with a medicament; and provide recommendations on whether a threshold dosage has been reached in relation to the neural biomarker response and/or provide recommendations on whether the patient is responding to the treatment on the basis of the neural biomarker response.

In an eight aspect, the present disclosure provides an apparatus for controlling one or more devices configured to provide a health-related recommendation for a patient, wherein the apparatus is configured to instruct at least one device to: identify a neural biomarker according to the second aspect and options; retrieve a set of neural signal data from the patient; determine a neural biomarker response for the identified neural biomarker based on the set of neural signal data; and provide a clinical recommendation based on the neural biomarker response according to the first aspect and options, and/or optimize a treatment based on the identified neural biomarker or for an illness, and/or recommend a pharmacological treatment based on a neural biomarker.

This application acknowledges that firmware and software can be valuable, separately tradable commodities. It is intended to encompass software, which runs on or controls “dumb” or standard hardware, to carry out the desired functions. It is also intended to encompass software which “describes” or defines the configuration of hardware, such as HDL (hardware description language) software, as is used for designing silicon chips, or for configuring universal programmable chips, to carry out desired functions.

The optional features or options described herein may be combined as appropriate, as would be apparent to a skilled person, and may be combined with any of the aspects of the invention.

### BRIEF DESCRIPTION OF THE DRAWINGS

Embodiments of the invention will be described, by way of example, with reference to the following drawings, in which:

FIG. 1a is a detailed pictorial diagram illustrating an example of a system for biomarker identification and treatment recommendation according to the present invention;

FIG. 1b is a detailed pictorial diagram illustrating an example of a system according to FIG. 1a;

FIG. 1c is another detailed pictorial diagram illustrating an example of a system according to FIG. 1a;

FIG. 1d is yet another detailed pictorial diagram illustrating an example of a system according to FIG. 1a;

FIG. 1e is yet another detailed pictorial diagram illustrating an example of a system according to FIG. 1a;

FIG. 2a is a flow diagram illustrating an example of a method for providing an assessment based on a neural biomarker response according to an aspect of the present invention;

FIG. 2b is a flow diagram illustrating an example of a method for identifying a neural biomarker based on neural signal data associated with an illness according to an aspect of the present invention;

FIG. 2c is a flow diagram illustrating an example of a method for applying neural biomarker response in clinical diagnosis and treatment screening according to an aspect of the present invention;

FIG. 3a is a schematic diagram illustrating an example of a system for optimizing a bioelectronic treatment for an illness according to an aspect of the present invention;

FIG. 3b is a schematic diagram illustrating an example of performing Bayesian optimization in order to optimize the bioelectronic treatment according to an aspect of the present invention;

FIG. 4a is a 2-D chart illustrating an example of at least one neural biomarker response according an aspect of the present invention;

FIG. 4b is another 2-D chart illustrating an example of at least one neural biomarker response according an aspect of the present invention;

FIG. 5 is a pictorial diagram illustrating an example of results from an optimizer configured to optimize neural response to treatment according an aspect of the present invention;

FIG. 6 is a block diagram illustrating an example of one or more system implement of the present invention;

FIG. 7 is a block diagram illustrating a computer or computing device suitable for implementing of the invention in relation to FIG. 6; and

FIG. 8 is a detailed pictorial diagram illustrating an example of clinical use environment (in surgery) according at least one aspect of the present invention.

Common reference numerals are used throughout the figures to indicate similar features.

### DETAILED DESCRIPTION

Embodiments of the present invention are described below by way of example only. These examples represent the suitable modes of putting the invention into practice that are currently known to the Applicant although they are not the only ways in which this could be achieved. The description sets forth the functions of the example and the sequence of steps for constructing and operating the example. However, the same or equivalent functions and sequences may be accomplished by different examples.

The inventive method, system, medium, and/or apparatus relates to neurally mediated therapies and/or the uses thereof, where at least one neural biomarker would be identified and its response measured, and applied applicably across a wide range of therapy areas. For a patient suffering from an illness, method, system, medium, and/or apparatus would provide suitable recommendations in relation to the neural biomarker(s) identified.

Illness herein refers to an unhealthy condition of the body or mind that a disease or inherited sickness may cause. Illness comprises various diseases and adverse symptoms susceptible to digital neural therapy. For example, diseases may be cardiovascular conditions or types of neural diseases.

The illness may be a neurally mediated illness in which the nervous system plays a key role in disease development, management, progression, etc. In general, neurally mediated therapies utilise bioelectronic neural stimulators or devices to treat such an illness. The bioelectric neural stimulators would simulate the nerves via electrical current. This simulation induce/block nerve signals, the effect of which impacts a target organ. Since the nervous system is an obvious mediator/controller of the organ and disease state, with relevance to cardiovascular disease, inflammatory conditions, etc., neurally mediated therapies would promote a therapeutically beneficial change in the patient's health or condition.

Neurally mediated therapies may include the use of one or more drug candidates that act via direct or indirect neural mechanism. Direct neural mechanisms include drugs like beta blockers, these block neural signals at the synapse, also newer classes of drugs like TRPA1/TRPV1 inhibitors that act to block the actions of external poisons, pain, heat etc (under development in e.g. asthma). Most anasthesia used molecules also fall into this class. This class is not just neural blockers, it also includes neural receptor agonists such as adrenaline which will induce sympathetic nerve activity.

Indirect neural mechanisms include drugs like nicotine which acts on receptors that modify other analytes that are directly neurally active. E.g. nicotine will reduce the levels of inflammation in the body by blocking cytokine release through Alpha-7 Nicotinic receptors. Cytokines are then detected in nerves and directly give rise to neural signals.

It is understood that the illness may have a defined set of conventional treatments and potential treatments. Potential treatments may include treatments undergoing clinical trials or testing.

Treatment herein refers to various invasive and non-invasive treatments targeting an illness. Treatment comprises one or more electrical stimulations to/of a nerve on the patient, administration of a medicament or medicine, and performance of surgical procedure(s) on the patient.

In the case of a treatment comprising one or more electrical stimulations to/of the nerve of the patient it is appreciated that changing the parameters of the electrical stimulation shall be construed as changing the applied treatment, this may include: the position on the nerve of the electrical stimulation (in any dimension such as radial, circumferential, longitudinal); changing the waveform of the electrical pattern applied to the nerve (including any parameters that may be used to represent a waveform such as amplitude, frequency, waveform shape, or otherwise); changing the timing of the electrical stimulation either in relation to the days it is applied, timing relative to the circadian cycle, timing relative to the administration of any other treatments (electrical stimulations, pharmacological, or otherwise), timing relative to natural biological processes such as the heart beat, blood pressure wave, respiratory cycle, gastric feeding/emptying cycle, or any other biological event.

The treatment may be accomplished or induced independently, or administered using the present BIOS Neural Biomarker Dosing & Exploration System. For example, the system may include a first device that performs electrical nerve stimulation (either invasively or non-invasively) and a second device that provides sensing and preferred dosing for the first device. In this way, it is appreciated by a person skilled in the art that the system need not be implemented in a single device and that the components of the system may be embodied separately, and each part of the system may be present for all or part of the patient's treatment.

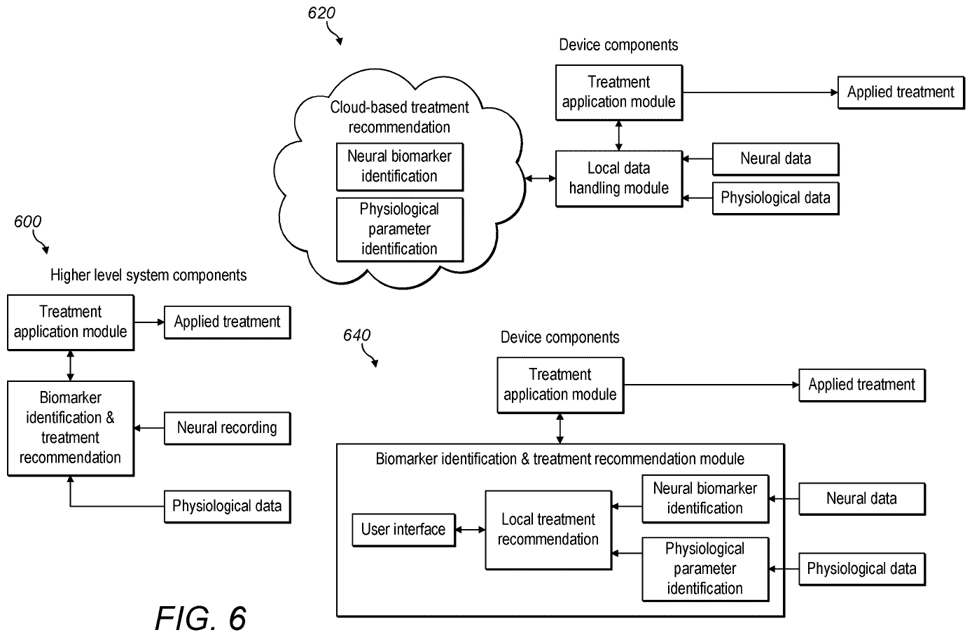

Further, the software and hardware of the system may be embodied together or separately on one or more devices either local or in the cloud as according FIG. 6. In one example, a patient is to receive an implantable vagal nerve stimulator (VNS) and during either implantation or clinical follow up a second device, the BIOS Neural Biomarker Dosing and Exploration System, is present alongside for the purpose of setting up and dosing the first device.

The treatment may further be a pharmacological compound and the system may be used to monitor, and assess the efficacy or other pharmacological criteria of a medicament when administered prior to the patient.

The treatment may be administered be in stages, for example, depending on the illness, there may be a period of baseline understanding prior to the treatment, then the initial treatment of a certain medicament, followed by continued use of another medicament following a surgical operation. The entire process or part of this process may constitute a treatment broken down into various stages of disease progression.

The BIOS Neural Biomarker Dosing & Exploration System, herein referred to as the “system”, may retain a set of neural signal data from the patient (suffering from the illness) in order to derive a neural biomarker response from the patient based on the treatment reference to healthy individuals absent of the illness. Various aspects of the “system” and the corresponding methods are described herein.

The set of neural signal data or neural signal data (used by the “system”) refers to a dataset collected and derived from neurological signals. This data can be collected directly or indirectly from the central and/or peripheral nervous system and processed thereafter by the system or separately before being inputted into the system. The processed data is decoded, with relevant information annotated or exacted, recorded/stored, and from raw electrical and chemical signals produced by the neurons of the nervous system. For example, the data may correspond to or depict a series of action potentials exhibited when the patient is/being treated. Neural signal data may include both endogenous and exogenous neural activity, that is, neural signals arising from internal, naturally occurring sources in the patient due to natural organ processes and functions of daily living, and neural signals that are created by the application of an external source such as electrical stimulation or the application of neurally-active molecules.

Neural data quality refers to the consideration of the inherent properties of the collection method in the process of deriving understanding and neural biomarkers from neural data. This includes consideration of the signal-to-noise ratio, other inherent noise sources in the usage environment, data buffering and handling to allow for local analysis for immediate usage, and sending some or all neural data to an offsite source (such as the cloud, or a dedicated higher CPU load computer) for deeper analysis including sending back updates to the local processing to improve locally processed data quality.

Further, neural data quality includes methodological considerations such as the type of modality or electrode used to collect the neural data. By way of example this could be the type of electrode used to collect the data which will naturally result in different data appearing but containing the same underlying information in the form of neural biomarkers. Consistent neural data quality can be considered to be achieved through a variety of methods, including classical data processing techniques such as filtering out noise sources, and feature engineering to derive specific (including spike extraction). Additionally, achieving neural data quality could also include methods such as deep learning to obtain stable feature extractions from noisy data or data of apparently different sources to get the same base neural information extracted.

Neural Biomarker refers to a sample of neural activity that is an objective measurement of a bodily variable, including biological processes, pathogenic processes, or pharmacologic responses to a therapeutic intervention, observed by monitoring one or several neural populations. Wherein the neural biomarkers represent objective indications of medical state.

Neural biomarkers are measured as features, in isolation, or linear or non-linear combination of features, of the acquired neural population activity, which may be calculated by processing the signals, or learned by one or more machine learning means, such as a machine learning processor. One or more machine learning models running on a machine learning processor may calculate neural biomarkers having been trained on data from one or more patients on the same part of the nervous system, or from a single patient over multiple time periods.

It is appreciated by a professional, skilled in the art, that the learned neural biomarkers may then be used as time and subject invariant stationary representations of the activity of the nervous system across a population of patients with the same indication, or for a single patient. Thus, a neural biomarker represents at least one repeatable feature from which the current neural activity can be understood as an indicator of a particular disease state or other physiological state and hence could be used as a basis for treatment decisions.

Neural biomarker additionally can refer to a naturally occurring molecule, gene, or characteristic in the brain or of the nervous system that serves as a sign of a normal or abnormal process, or of a condition or disease. The neural biomarker may comprise known and unknown biomarkers, with known disease-related biomarkers such as Neurofilament Light (NfL), Tau, GFAP; or informative markers that may be detected at much earlier stages, in blood, serum or plasma, enabling a better understanding of the long-term effects and disease pathology without invasive measures.

Neural biomarker response(s) refers to the response of the neural biomarker shown as a change in the neural signal induced by a treatment or when one or more physiological parameters are adapted. The neural biomarker response serves as the output the system used in a gamut of medical applications, such as providing treatment recommendations. Provided that upon, during, or following (in response to) the treatment, there would be at least one change to the neural biomarker data representative of neural biomarker response within this time period to which the clinicians may use the neural biomarker response or the underlying data as the basis for deciding which treatment would be applicable.

It is appreciated that Neural Biomarkers are features of neural data and that the biomarker may be considered to be the rate of change of a feature of neural signal data, or any other sub processed feature which may or may not relate to a human observable quantity. As such the Neural Biomarker response, and general statements such as, “a change from the baseline” of a neural biomarker should be understood in the relative context as relating to any measurable difference and need not be related to the specific context of the change in absolute value of a quantity.

Neural fibre type refers to the naturally occurring different types of nerve fibre that exist in humans and other species according to the Erlanger and Gasser classification, e.g. A-alpha, A-beta, A-gamma, A-delta, B, C fibres. These neural fibre responses may be easily identified by their differing conduction velocities. Further it is understood that neural tissue is not a homogenous structure and this property can be used to classify nerve fibres and neural data into biomarkers: in the case of the brain different areas of neural tissue have specific functions and hence neural activity can be turned into a biomarker based on properties of underlying neural signal data patterns as well as the location these patterns occur in the brain; in the peripheral nerves individual nerve fibres are grouped into fascicles which, dependent on the anatomical location, may map to one organ or another preferentially, as such nerve fibre types in a specific location in the nerve may be different neural biomarkers of different neurally-mediated processes. Hence, neural fibre type or classification could refer to any other fibre type classification or method of separating any population of nerve fibres from another population that is known to a person skilled in the art.

A physiological parameter may be derived/deduced from physiological data collected from the patient before, during, and after the treatment. The physiological parameters may be derived or estimated from one or more machine learning models (simulating the disease state of the patient) that is trained using physiological data. Other methods for obtaining these parameters may also be available such as taking direct measurements from the patient.

Threshold dosage refers to a value or a combination of dosage values reflecting a certain level of efficacy for the patient undergoing the treatment, or when the treatment meets one or more other pharmacological criteria with respect to the patient's physiology or physiological parameters. The value(s) may be quantitative and used for assessing how much treatment is necessary for the patient. The threshold dosage serves as an indication of which the treatment may be selected or abandoned otherwise. The threshold dosage may be used to provide dosing reference(s) for certain types of treatments. With respect to the neural biomarker response to treatment, the threshold dosage may be a historical indicator for recommending such treatment and if necessary by what amount. It is understood that the pre-determined threshold dosage may therefore be updated based on the neural biomarker response or a change in the response thereof. The threshold dosage may refer to the therapeutic efficacy of a treatment, or it may equally be understood to refer to the tolerable threshold of a side effect of the treatment. Further, a threshold dosage may equally be considered to be understood as achieving the addition of a new effect, or the subtraction of an existing one.

Furthermore, threshold dosage may consider the safety or device type in its determination: this could be by means of not exceeding a certain charge injection per unit time in order to consider tissue safety limits; or consideration of the effect of the applied dosage on the device such as whether the applied stimulation current may cause the device to run out of power too quickly, or heat up too much.

Schedule of the treatment or treatment schedule includes the type of treatment that will be given, how it will be given, and how often it will be given. The schedule may be curative, palliative, and/or preventative in nature. The schedule may be determined or adjusted from a predetermined treatment plan based on the neural biomarker response provided by or from the system.

Physiological data refers to data collected from the patient directly or indirectly in relation to the illness. The data may be representative of a physiological response, such as a response exhibited by patient during the onset of the disease or following a treatment. The data may include, for example, standard parameters such as blood pressure, heart rate, breathing rate, diastolic pressure change, systolic pressure change, temperature, and other physiological parameters associated with the body. Further physiological parameters may be further derived or generated using one or more machine learning models trained on the physiological data in relation to the health state or condition of the patient. Further, the physiological data may be equally obtained by a physiological sensor, or subjectively inferred by the observation of a trained clinician. The physiological data may be part of, be combined with, representative of other data in relation to the patient.

Machine learning models may apply one or more machine learning techniques such as an algorithm/method that can be used to generate a trained model based on labelled and/or unlabelled training datasets; one or more supervised ML techniques; semi-supervised ML techniques; unsupervised ML techniques; linear and/or non-linear ML techniques; ML techniques associated with classification; ML techniques associated with regression and the like and/or combinations thereof. Some examples of ML techniques/model structures may include or be based on, by way of example only but are not limited to, one or more of active learning, multitask learning, transfer learning, neural message parsing, one-shot learning, dimensionality reduction, decision tree learning, association rule learning, similarity learning, data mining algorithms/methods, artificial neural networks (NNs), autoencoder/decoder structures, deep NNs, deep learning, deep learning ANNs, inductive logic programming, support vector machines (SVMs), sparse dictionary learning, clustering, Bayesian networks, types of reinforcement learning, representation learning, similarity and metric learning, sparse dictionary learning, genetic algorithms, rule-based machine learning, learning classifier systems, and/or one or more combinations thereof and the like.

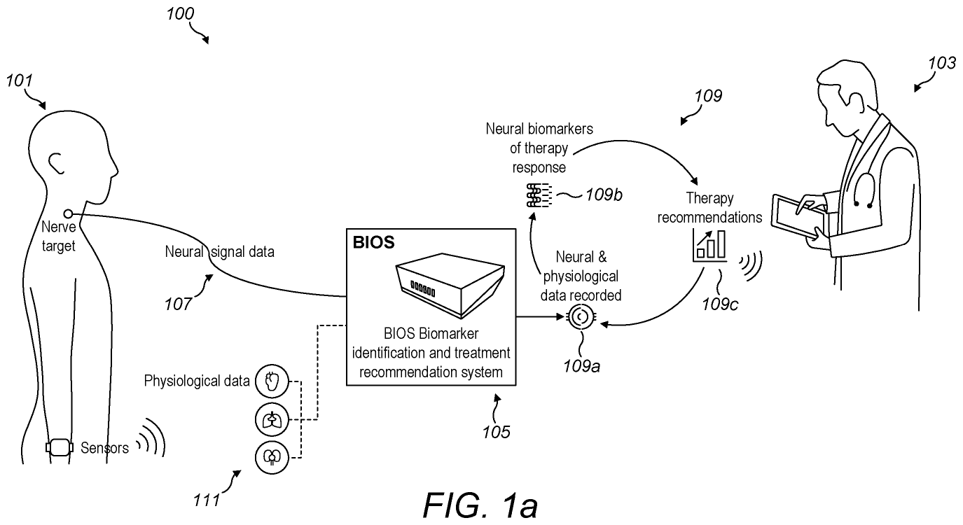

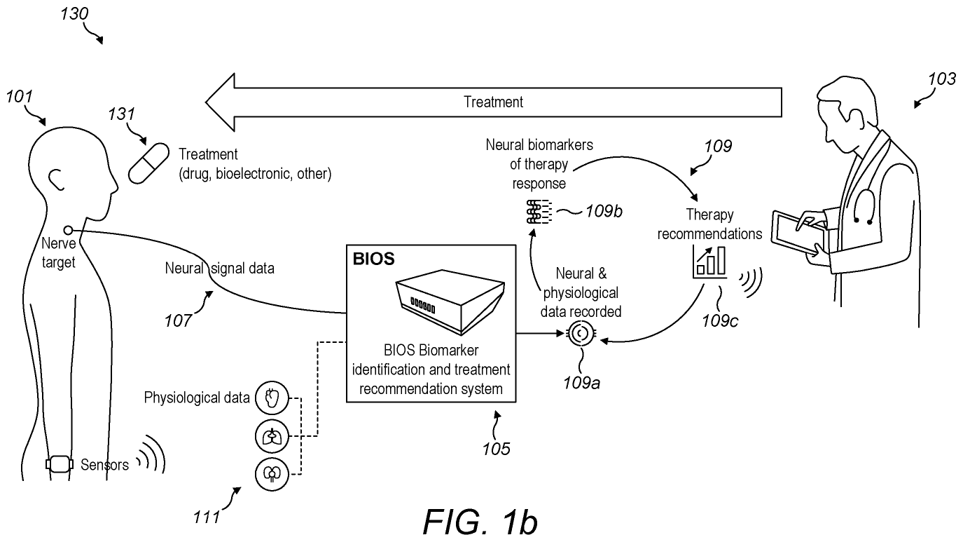

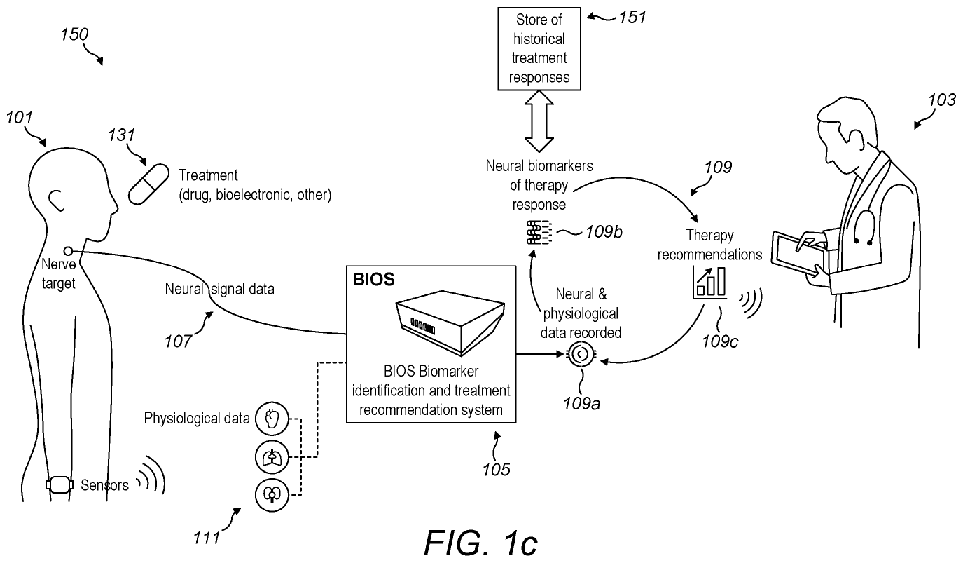

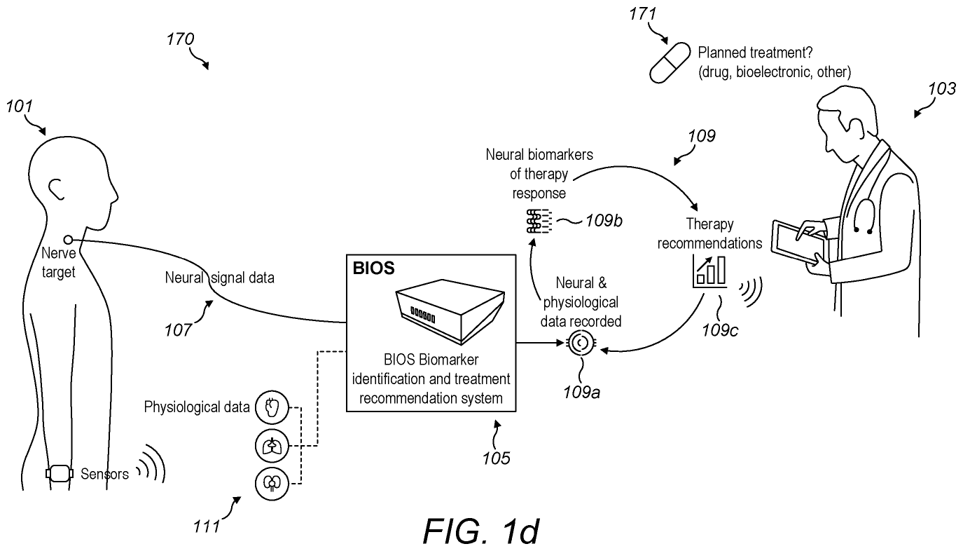

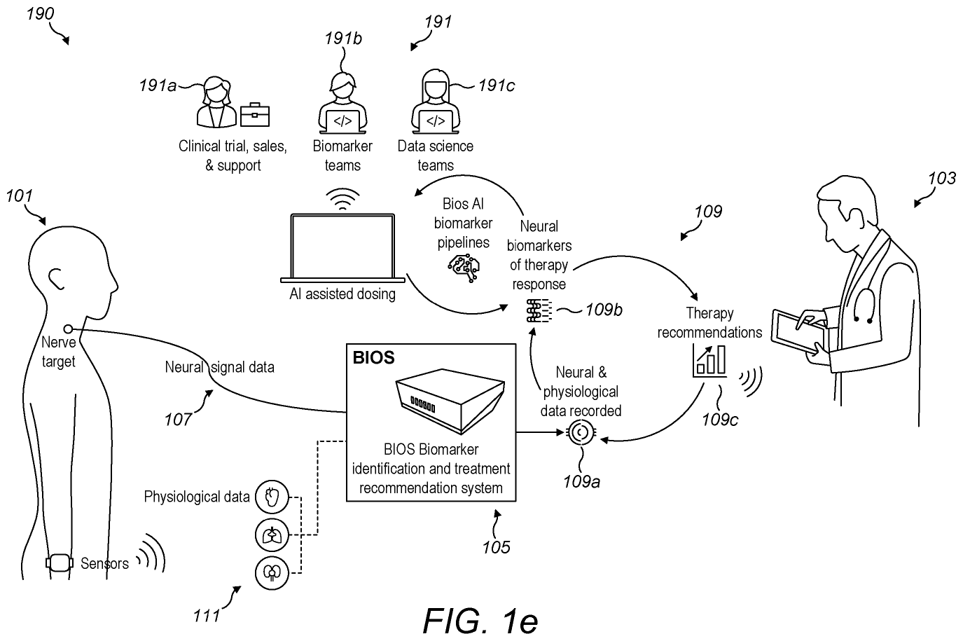

FIGS. 1a to 1e 100, 130, 150, 170, 190 each provide a detailed pictorial diagram illustrating an example of a system 105 for biomarker identification and treatment recommendations and the use of the system thereof. BIOS Neural Biomarker Dosing & Exploration System 105, otherwise referred to as “system”, provides one or more recommendations (that may be clinically relevant) with respect to the patient's health or ill-health. The recommendation may be a recommendation concerning one or more treatments for an illness based on the neural biomarker response.

In these figures, the neural signal data 107 may be collected from a human 101, where the human 101 may be in ill-health or is a patient suffering from a disease or illness. The human may use a wearable device such as a smartphone to transmit physiological data 111 via a sensor. The observation of a trained clinician may subjectively infer the physiological data 111. Such physiological data may be part of, be combined with, or representative of other data in relation to the patient.

As shown in FIG. 1a, according to one or more aspects of the present invention, including BIOS Neural Biomarker Dosing & Exploration System 105, is configured to receive the neural signal data 107 and determine a baseline for the neural biomarker response from the neural signal data 107 in the absence of the treatment or other forms of action (for the patient) that results in recorded neural activity/neural signal data 107, and/or physiological data 111. Using the baseline determination, system 105 can provide an assessment 109 of the patient's condition following treatment or therapy. Specifically, system 105 is able to: 1) monitor/record the physiological data 111 based on the neural signal data 107, 109a; 2) determine the neural biomarker of the therapy response 109b; and 3) provide therapy recommendation 109c. The assessment 109 is provided to a physician 103 to be further considered before any treatment has been administered. Depending on the treatment, the system 105 will accommodate Further shown in FIG. 1b, system 105 provides the assessment 109 for a physician to consider following one or more treatments. The system 105 continues to monitor the neural activity/neural signal data 107 based on the physiological data 111. The treatment may be a drug, bioelectronic stimulation, or other medium that causes the human 101 to exhibit above baseline neural activity/neural signal data 107 in relation to the physiological data 111.

In FIG. 1c, system 105 may be configured to store or use a database of historical records 151, i.e. a store of historical treatment response. This data may continuously update the assessment 109 such that the neural biomarker of the therapy response 109b may be re-evaluated. The stored assignment may form a reference set for planning or adjusting the treatment according to FIG. 1d.

In FIG. 1d, system 105 may be configured to change treatment deposition based on the recorded physiological data 111 and the neural signal data 107. System 105 can compile the responses received to form a reference set, where the set can be used to generate a personalised neural biomarker dose response. Any previously planned treatment 171 can thus be updated for the patient based on the personalised neural biomarker dose response such that the appropriate treatment results can be achieved using system 105.

Finally, in FIG. 1e, system 105 can interact and transmit data for use by external parties 191 in confidence in a secure manner. These parties may include but are not limited to clinical trial personnel, sales, and support members in drug discovery 191a, biomarker investigator 191b, and data scientists 191c. These parties may help curate the data and develop BIOS Neural Biomarker Dosing & Exploration System, which includes an Artificial Intelligence (AI) pipeline 192 using one or more machine learning models described herein for drug dosing using one or more machine learning models described herein. Following are some example systems that are part of one or more aspects of the present invention.

In one example, the system receives a set of neural signal data; determine a baseline for the neural biomarker response from the set of neural signal data in the absence of the treatment; administer a treatment based on a schedule to the patient while obtaining the set of neural signal data collected from the patient; identify one or more changes in the set of neural signal data in response to and for a duration of the treatment; determine the neural biomarker response based on said one or more changes in the set of neural signal data through the time period appropriate to the treatment; ascertain the neural biomarker response in relation to the baseline; provide an assessment of the treatment based on the neural biomarker response; and output the assessment as the recommendation. Here the clinician would be using the neural biomarker to recommend the treatment or an alternative treatment based on the neural biomarker response present with the treatment.

In another example, the system receives a set of neural signal data; determine a level of the neural biomarker present with respect to a historical patient population with the illness and/or patient specific levels of the neural biomarker associated with the illness; determine a state or severity of the illness based on one or more determined levels of the neural biomarker as compared to the historical patient population or the patient specific levels of neural biomarker characteristics (i.e. measure the amount of circulating cholestorol, look at the LDL and HDL cholesterol levels) associated with the illness; provide an assessment of one or more treatments based on said determinations, wherein the assessment correlates one or more neural biomarker levels in relation to those of, or known for, the illness; and output the assessment as the recommendation.

Here the clinician would be using the neural biomarker in the classical way a blood analyte is used to diagnose an illness. The measurement is taken; they compare its amount to some pre-known level and say whether you have the disease. E.g. measure the amount of circulating cholesterol, and look at the LDL and HDL cholesterol levels. Tell you whether or not you have progressing heart disease or not.

In another example, receive the set of neural signal data; determine, in the absence of treatment for the illness, the neural biomarker response for an expected response of a treatment based on a patient population with the treatment and/or historical levels of the neural biomarker response from a different patient in response to the treatment; provide an assessment of one or more treatments based on the determination in relation to the neural biomarker response historically known for the illness; and output the assessment as the recommendation.

Here the clinician would be using the neural biomarker to assess whether a patient is a likely responder to a certain treatment without actually applying the treatment. e.g. they record the neural biomarker associated with an overactive renal sympathetic nerve and if they see it is overactive then they may choose to provide a sympathetic nerve activity reduction treatment (drug such as beta blocker, surgical procedure such as ablation).

The following options are provided in relation to the system. It is understood that these options may be combined as appreciated with other features described herein. Other options or optional features in relation to methods described herein may also be combined with the system to provide the benefits highlighted in the previous sections.

In one option or optionally, the assessment provided by the system comprises one or more proposed modifications to a schedule of the treatment. Optionally, an external device is configured to collect the set of neural signal data in a continuous and uninterrupted manner. Optionally, the set of neural signal data is collected and stored on an external device. Optionally, the set of neural signal data is collected for a time interval. Optionally, the time interval begins from a first time point before a treatment is administered and ends at a second time point after the treatment has been administered.

Optionally, the set of neural signal data is obtained in real-time as a treatment is being administered. Optionally, the set of neural signal data is obtained from one of: an implantable neurally-connected device with continuously recording functionality; an implantable neurally-connected device that records neural data either in batches or only when powered or wirelessly connected to a second external device; a neurally-connected device that temporarily goes through the skin to a nerve for the duration of recording; a neurally-connected device that is temporarily situated inside a patient and can be connected to by a second device for periods of recording; a fully non-invasive neural device that detects neural activity wirelessly at a distance from the nerve; a system that detects neural activity based on a prior procedure to allow detection; a device with a system that is configured to detect non-neural signals that themselves contain proxies of neural data; or a device employing one or more methods of detecting neural data or proxies of neural data.

Optionally, the set of neural signal data comprises time series data capturing different treatment stages in relation to one or more physiological responses exhibited by the patient during the different treatment stages. Optionally, each treatment is selected from a set of available treatments based on a physiological condition of the patient in relation to a determined progression of the illness. Optionally, each treatment is further selected from a set of available treatments based on the neural biomarker response of the patient in relation to the treatment and/or the illness.

Optionally, the system further comprising: a set of treatments associated with the illness, wherein each treatment of the set is trialled with a patient and a preferred treatment is selected based on the neural biomarker response of the patient in relation to the set of treatment and/or the illness.

Optionally, said one or more treatments comprise bioelectronic therapies and/or molecular therapies. Optionally, the bioelectronic therapies comprise an electrical stimulation of a nerve of the patient. Optionally, the treatment is a bioelectronic treatment and the neural biomarker response is between 0.5 ms to 5 ms following the treatment. Optionally, the treatment is a bioelectronic treatment and the neural biomarker response is between 5 s to 60 s following the treatment. Optionally, the molecular therapies comprise an administration of a medicament to the patient. Optionally, the treatment is a molecular treatment and the neural biomarker response is between 1 s to 180 s following the treatment. Optionally, the treatment is a molecular treatment and the neural biomarker response is between 0.5 hr to 4 hr and/or 1 hr to 24 hr following the treatment. Optionally, the illness is a respiratory inflammatory illness.

Optionally, said one or more changes exhibited in the set of neural signal data are statistically significant in relation to the set of neural signal data at a time point before the treatment is administered. Optionally, said one or more changes correspond to at least one change or improvement in one or more physiological parameters of the patient with respect to the treatment.

Optionally, the neural biomarker response is induced by the onset, or progression, of the illness and/or the treatment. Optionally, the illness comprises a neurally-mediated and/or neural-related disease and/or condition. Optionally, the assessment comprises a schedule of a treatment that follows a pre-determined threshold dosage that is set based on historical treatment data associated with illness.

Optionally, said one or more modules are configured to: measure the neural biomarker response at a time point following treatment; adjust a treatment based on whether the measured response at the time point meets a pre-determined neural biomarker response threshold; modify a schedule of the treatment in relation to the adjustment, wherein the schedule is adapted to include the adjustment based on a pre-determined treatment plan; and output the schedule of the treatment.

Optionally, said one or more modules are configured to: determine one or more physiological parameters of the patient prior to initiating the treatment; identify a treatment based on said one or more physiological parameters; initiate the treatment to elicit the neural biomarker response; and adapt the treatment based on a change in at least one physiological parameter in relation to the neural biomarker response.

Optionally, said one or more modules are configured to: measure the neural biomarker response of a treatment for a time interval; and update a pre-determined neural biomarker response threshold based on the measurement.

Optionally, the system further comprising: at least one module that is configured to: provide one or more types of electrical stimulation to a nerve of the patient exhibiting the treatment. Optionally, the system further comprising: at least one module that is configured to: induce the treatment based on a change in a physiological parameter of the patient in relation to the illness. Optionally, the neural biomarker is used to guide dosing of a treatment.

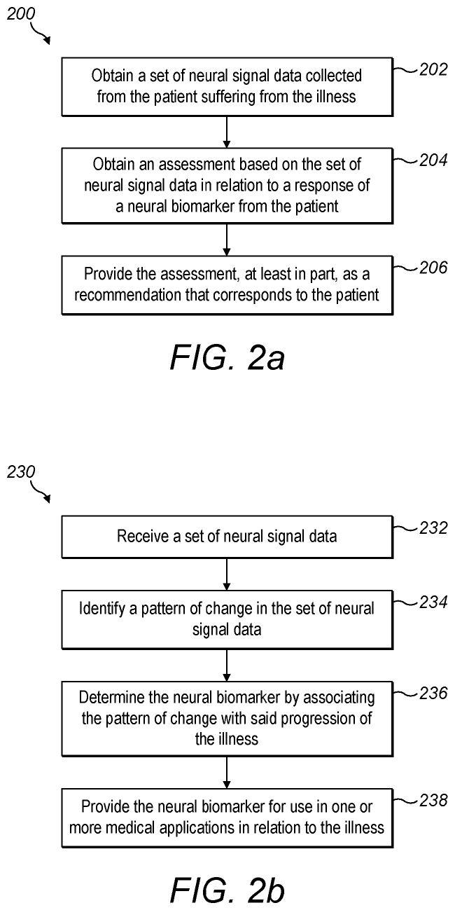

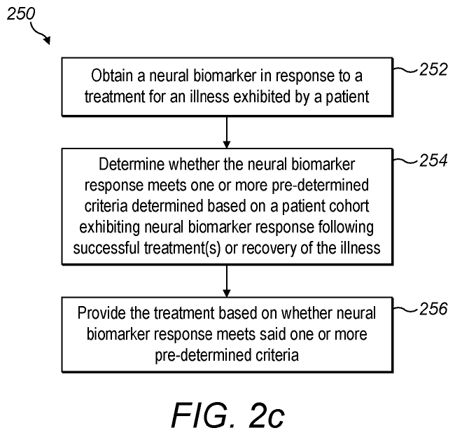

FIGS. 2a to 2c present flow diagrams of method steps that would be understood by the skilled person to be suitably used in conjunction with or as part of the system herein described. Such a system may further comprise an external device for obtaining the set of neural signal data used by the method.

FIG. 2a illustrates an example of a method for providing an assessment based on a neural biomarker.

In step 202, obtaining a set of neural signal data collected from the patient suffering from the illness, wherein the set of neural signal data is collected with respect to a neural biomarker; in step 204, obtaining an assessment based on the set of neural signal data in relation to a response of a neural biomarker from the patient, wherein the biomarker response is indicative of one or more determinations made with respect to the neural biomarker by the system; and in step 206, providing the assessment for the patient.

FIG. 2b illustrates an example of a method for identifying a neural biomarker based on neural signal data associated with an illness.

In step 232, receiving a set of neural signal data, wherein the set of neural signal data is collected from a patient during a progression of the illness; in step 234, identifying a pattern of change in the set of neural signal data; in step 236, determining the neural biomarker by associating the pattern of change with said progression of the illness; and in step 238, providing the neural biomarker for use in one or more medical applications in relation to the illness.

Optionally, administering a treatment for the illness to the patient; receiving a set of neural signal data collected from a patient a period following the treatment; comparing the pattern of change following the treatment with that in the absence of the treatment; identifying a change with respect to the treatment in the pattern of change in the set of neural signal data; an ascertaining the neural biomarker based on the identified change.

Optionally, the pattern of change is associated with at least one deviation of: signal data collected from the patient, historical neural signal data of the patient, historical neural signal data associated with a population of patients, a historical neural signal data of a non-human subject with the illness and/or being administered the treatment.

Optionally, further comprising: receiving a set of physiological data corresponding to the set of neural signal data; identifying the initial pattern of change in the set of neural signal data based on historical neural signal data obtained from a population without the illness; and confirming the neural biomarker based on the pattern of change in relation to the set of physiological data, wherein the set of physiological data is at least partially indicative of said progression of the illness without treatment.

Optionally, further comprising: selecting a treatment for the illness; measuring a response to the identified neural biomarker based on the treatment with respect to the pattern of change identified; and compiling the response as part of neural biomarker responses associated with the illness.

Optionally, further comprising: obtaining one or more responses for the identified neural biomarker; compiling said one or more responses as a reference set, wherein the reference set is structured with respect to or to reflect said progression of the illness for the patient, for a sub-population of patients associated with the patient, or for a cohort of patients suffering from the illness; providing a personalised neural biomarker dose response based on the reference set; and updating a treatment plan for that patient based on the personalised neural biomarker dose response, wherein the treatment plan comprises at least a dosage plan.

Optionally, further comprising: selecting the treatment in relation to said one or more medical applications; and adapting the treatment based on the neural biomarker responses. Optionally, further comprising: performing said one or more medical applications using the neural biomarker; and adapting said one or more medical applications in accordance with a response from the neural biomarker. Optionally, further comprising: obtaining one or more responses for the identified neural biomarker; compiling said one or more responses as a set of historical neural biomarker responses that is used as the baseline in the system, according FIGS. 1a to 1e, for the patient or a population of patients with the illness that has been administered with that treatment; and providing the assessment from said system with respect to and in consideration of the set of historical neural biomarker responses.

Optionally, further comprising: providing the neural biomarker to be used with an algorithm; and allowing the algorithm to update and/or adapt the treatment or a treatment plan towards a target therapeutic objective, wherein the target therapeutic objective is defined on the basis of a target neural biomarker, a target physiological response, a clinician-defined observation, and/or a combination thereof, wherein the algorithm is configured to optimize for the target therapeutic objective based on the set of neural signal data. Optionally, the algorithm is configured to: optimize periodically with respect to days, weeks, or months as the treatment plan is being or to be updated with respect to a long-term target therapeutic objective.

In relation to the above, it is understood that the target therapeutic objective is the “threshold dosage” that the algorithm is trying to reach (including combination of achieving efficacy and avoiding side effects). This target therapeutic objective may change as the treatment is applied or disease progresses. The manner in which the target amount of therapy changes may be determined by observation of the patient over time or it may be pre-known for a specific treatment or disease that the response to treatment will change in a certain way and hence the long term target will change E.g. A treatment (bioelectronic or molecular) is applied and optimised to a specific dosage on day 1, this dosage either adequately addresses the illness, or may be a safe test amount the clinician prefers, or the amount of treatment given is limited by a side effect.

Subsequently one of a few things may happen: 1. The disease starts to get better and the amount of treatment required to maintain the patient at a healthy level goes down; 2. The patients disease gets worse or changes due to other environmental factors (age, diet etc) and the amount or combination of treatments must be increased/modified; 3. The patient becomes habituated to the treatment (either the response drops off or the side effects start to reduce) the amount of treatment must be increased in order to get the same therapeutic effect or can be increased now that side effects have subsided.

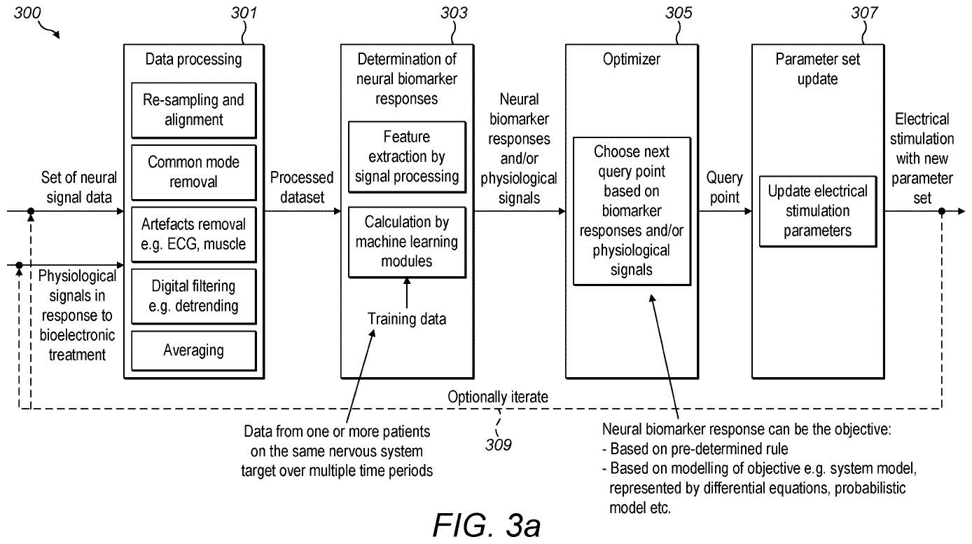

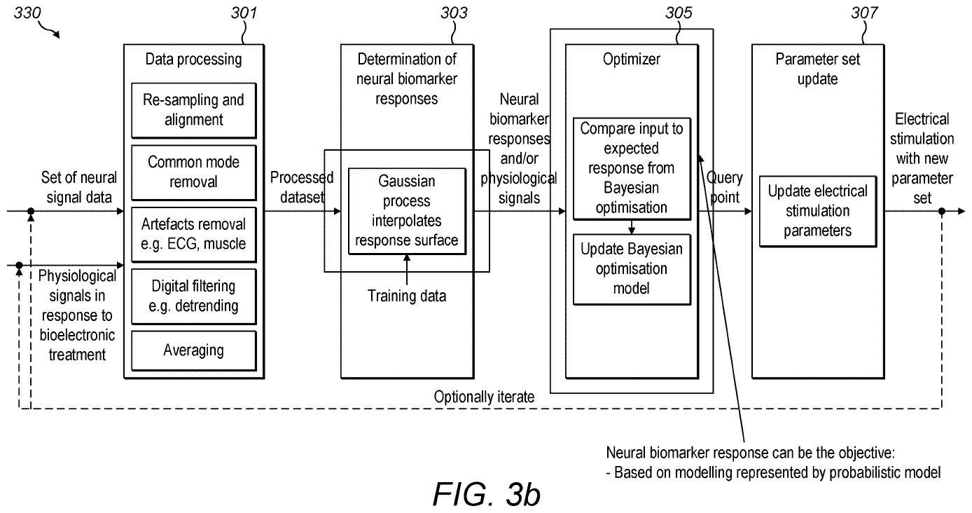

It is an option and further understood that the algorithm may comprise one or more Bayesian optimization techniques. The application said one or more Bayesian optimization techniques are further illustrated in FIG. 3b. Detailed implementation of the Bayesian optimization in relation to the vagus nerve stimulation is provided by the document “Online Bayesian Optimization of Nerve Stimulation”, Lorenz Wernisch et el., https://doi.org/10.1101/2023.08.30.555315, as incorporated herein by reference and referred to as “Document”.

The implementation described in the Document leads to the development of the neural biomarker dosing system described herein. For the case where a VNS stimulation is being optimized by a system to pick a preferred treatment, the neural signal may be recorded from the vagus nerve and is used in combination with physiological signals to provide neural biomarkers. The treatment may be an electrical stimulation applied to the nerve. The system may pick the preferred treatment using a machine learning technique or a machine learning based optimization model. The machine learning based optimization model specifically described in the Document is a Bayesian optimization model.

FIG. 2c illustrates an example of a method for applying neural biomarker response in clinical diagnosis and treatment screening.

In step 252, obtaining a neural biomarker response in response to a treatment for an illness exhibited by a patient; in step 254, determining whether the neural biomarker response meets one or more pre-determined criteria determined based on a patient cohort exhibiting neural biomarker response following successful treatment(s) or recovery of the illness; in step 256 and providing the treatment based on whether neural biomarker response meets said one or more pre-determining criteria, wherein the treatment is used in relation to the clinical diagnosis and treatment screening.

Optionally, the biomarker response is obtained for a neural biomarker identified. Optionally, said one or more pre-determined criteria are associated with at least one physiological parameter of the patient. Optionally, clinical diagnosis and treatment screening comprise one or more stages of the clinical trials.

Optionally, the method further comprising: generating a schedule of the treatment based on the neural biomarker response, wherein the schedule is formulated in relation to one or more physiological parameters of the patient following the treatment.

FIG. 3a is a schematic diagram illustrating an example of a system for optimizing a bioelectronic treatment for an illness 300. In the figure, various stages of the present invention are highlighted, where the input data (set of neural signal data and physiological signals in response to bioelectronic treatment) traverse these stages when received by the system. The stages include data processing 301, determining neural biomarker response(s) 303, optimization 305, and parameter update 307 with optional iteration 309.

In the data processing stage 301, the system employs various methods, including but not limited to re-sampling and alignment, common mode removal, artifact removal, digital filtering, and averaging. Following processing, the system proceeds to determine 303 the neural biomarker response(s) associated with the processed input data. This input data can originate from one or more patients, all targeting the same nervous system, across multiple time periods. The system's objective may be to determine the responses based on this input data. For instance, it can utilize feature extraction through signal processing or employ one or more pre-trained machine learning models. The training of the machine learning model can be done within the system using one or more sets of training data fit for the purpose.

Using the physiological data and/or biomarker responses as part of the input, the next stage, optimization 305, requires an optimizer, as described in the following section. This optimizer offers a convenient means for the system to update or fine-tune parameters (307) based on a specific objective-oriented query point or query point with respect to the underlying data read by the system. The query point may be time dependent. The query point is chosen based on the physiological data and/or biomarker responses as well as the objective. The query point determines when to update the electrical stimulation parameters of the system and by what amount. The system may apply the updated parameters to initiate the next iteration of electrical stimulation. In stages 305 and 307, the input data passes through one or more models or search algorithms, with evaluations based on achieving a particular objective. This objective guides the determination of a query point, which is then used to update the set of electrical stimulation parameters. This entire process, encompassing stages 301-307, may iterate until the desired objective is attained.

In one aspect of the system, the system comprises one or more modules configured to: apply an electrical stimulation to a nerve of the patient based on a set of electrical stimulation parameters; obtain a set of neural signal data from the patient in relation to the electrical stimulation; determine one or more neural biomarker responses from changes in: the set of neural signal data indicative of changes in neural activity, wherein the changes are associated with the progression of the illness, in response to the bioelectronic treatment, or when undergoing the bioelectronic treatment, or for a period following the bioelectronic treatment, or a combination thereof; obtain one or more physiological signals of the patient in response to the bioelectronic treatment; and transmit said one or more neural biomarker responses and/or one or more physiological signals to be analysed by an optimizer, wherein the optimizer is configured to: receive said one or more neural biomarker responses and/or said one or more physiological signals as input data to a model or search algorithm related to the model; evaluate the input data based an objective; determine a query point based on the objective; and output the query point; updated the set of electrical stimulation parameters based on one or more outputs from the optimizer.

The optimizer is configured to maximised or minimised the neural biomarker, combination of Neural Biomarkers or combination of Neural Biomarkers and physiological data. According to the Document, the objective function to be maximised or minimised may be any combination of the biomarkers (e.g. maximum baroreflex activation while minimising Neural Biomarkers of breathing effects).

In one example, steps performed by the optimiser include comparing the neural and physiological biomarkers to a model of the neural and physiological expected response (which may be based on a system model, may be represented by differential equations, may be a probabilistic model); and update the model based on the new data; produce a new query point based on the models recommendation; and update the set of electrical stimulation parameters to the query point. In another example, the optimization algorithm may be a search algorithm optimizer that evaluates the values of the neural and physiological biomarkers and produce a new query point according to a predetermined rule or rule set. For both examples, the optimizer is used to update the set of electrical stimulation parameters to the query point.

Optionally, said one or more neural biomarker responses comprise at least one response of the neural fibre types to electrical stimulation of the nerve. Optionally, said one or more neural biomarker responses comprise at least one response of neural fibres within different spatial locations of the nerve. Optionally, said one or more neural biomarker responses comprise measurements of a neurally-mediated process. Optionally, the updated set of electrical stimulation parameters is delivered automatically by a device configured to implement the model. Optionally, the system for optimizing the bioelectronic treatment would run through multiple iterations and the electrical stimulation parameters are updated for each iteration. Optionally, the updated set of electrical stimulation parameters is reviewed and/or modified by a clinician. Optionally, the updated set of electrical stimulation parameters is processed by a second model to assess for safety.

In one example, the second model could be a simple piece of data processing of absolute values. Or a more complex set of evaluations. For instant evaluating the electrical stimulation parameters to see if a single parameter, or combination of parameters, does not go above any predetermined thresholds or does not step up too much in one step. e.g. These include: 1. Current not greater than X amps; 2. Total charge (current * pulse width) not greater than Y Coulombs; 3. Step change in parameters not greater than Y amount different to the prior set; The X, Y, Z limits may be set based on known physiological safety, device parameters, or at a clinicians preference.

Optionally, the set of electrical stimulation parameters is associated with square wave stimulations comprising one or more of: a current, a pulse width, a train frequency, a polarity, a train duration, a duty cycle on/off time, a spatial location. Optionally, the set of neural signal data is controlled for neural data quality, and the optimization takes into account the neural data quality during each update.

In one example, the optimiser may use a different form of model and model initialisation parameters dependent on the underlying noise sources of the data or other features of the data type. N.B; herein covered is the concept of neural data quality in the description as both noise and the modality of collection.

Optionally, the system furthering comprising: optimizing the bioelectronic treatment using Bayesian optimization based on one or more electrical stimulation parameters, wherein the bioelectronic treatment comprises at least one vagus nerve stimulation. This further described according to the Document.

Optionally, wherein the updated set of electrical stimulation parameters is used for training at least one model configured to: identify one or more changes in a set of neural signal data indicative of a neural biomarker response; and ascertain the neural biomarker response based on said one or more changes for the duration of the treatment.

Optionally, the system further comprising: applying the updated set of electrical stimulation parameters to obtain a set of neural signal data collected from the patient before, during, and after the treatment. Optionally, further comprising: applying the set of neural signal data to the system to obtain a recommendation based on the updated set of electrical stimulation parameters.

FIG. 3b is a schematic diagram illustrating an example of performing Bayesian optimization in order to optimize the bioelectronic treatment according to the Document. Stages 303 and 305 differ from that of FIG. 3a. In Stage 303, a Gaussian process may be applied to determine the relevant neural biomarker response(s). In stage 305, the optimization process requires a Gaussian optimization model, which compares the input to the expected response(s) in the previous stage. The Gaussian optimization model is thereby updated based on the comparison. This process is further described in the Document. The final output (updated electrical parameters of the system) from the adjustment in stages 303 and 305 remains the same as that of the above according to FIG. 3a.

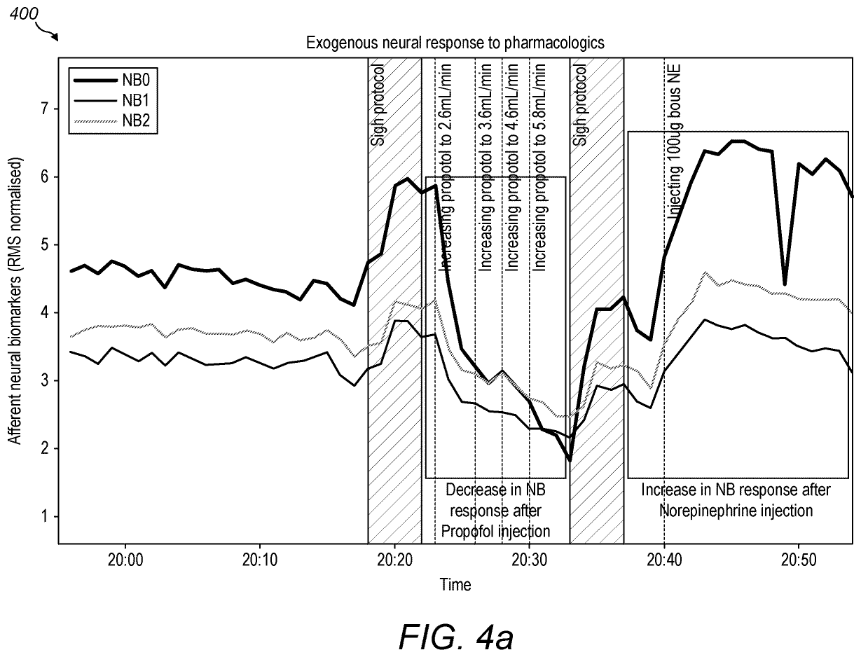

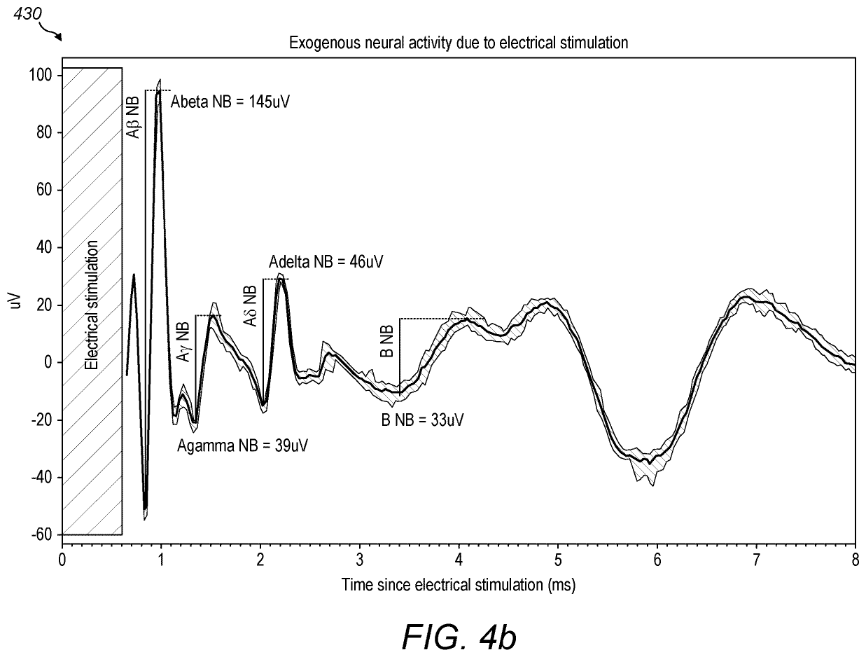

FIGS. 4a and 4b are 2-D charts illustrating an example of at least one neural biomarker response. FIG. 4a shows exogenous neural responses (with respect to biomarkers NB0, NB1, and NB2) to pharmacologics, norepinephrine and propofol. The exogenous neural responses are recorded by the system for each biomarker. These pharmacologics are administered via injection following standardised protocols at varying dosages and times. The responses are normalized as shown by the y-axis of the figure. FIG. 4b shows exogenous neural responses to electrical stimulation. Both figures are plotted with respect to time.

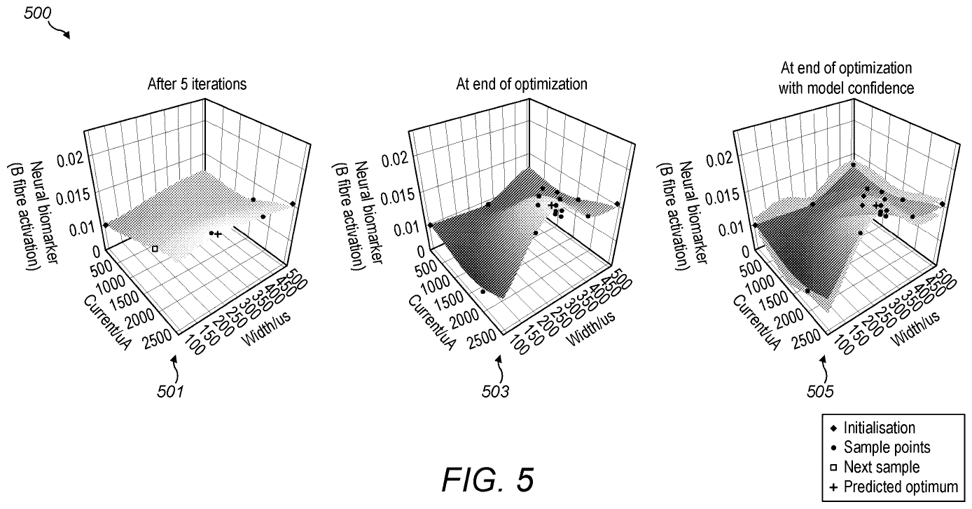

FIG. 5 provides a visual representation of results obtained from an optimizer designed to enhance neural response to treatment. The figure plots (in a 3-D chart) the input data and showcases the query points, including the initialization, previously sampled, next, and predicted optimum points. Here the algorithm optimizes the objective of “maximizing B-fibre activation while minimizing current amplitude” within a model. The model assesses the objective function across the input space using input data and identifies the next query point for sampling, as depicted. The subsequent query points. The estimated optimum (displayed on the left) after five iterations 501. The estimated optimum at the end of the optimization procedure (shown in the middle) 503. The model's confidence level in the estimation provided by the objective function (demonstrated on the right) 505.

FIG. 6 is a block diagram illustrating an example of one or more system implementations. The system may be implemented on a single or multiple devices, where each device comprising: at least one component configured to: identify a neural biomarker for use with the system based on a method described herein. The figure shows various set up for the system, ranging from a single device 600 to part of multiple devices 620, 640.

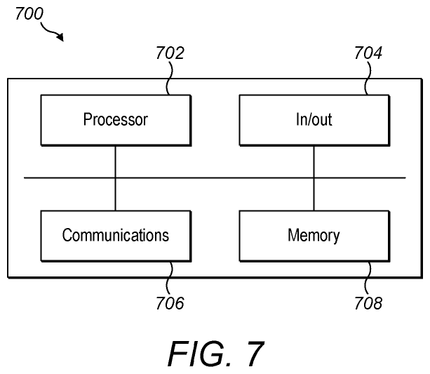

FIG. 7 is a block diagram illustrating a computer or computing device suitable for implementing of the invention in relation to FIG. 6. The figure shows an example computing apparatus/system 700 that may be used to implement one or more aspects of the system(s), apparatus, method(s), and/or process(es) combinations thereof, modifications thereof, and/or as described with reference to the previous figures and/or as described herein. Computing apparatus/system 700 includes one or more processor unit(s) 702, an input/output unit 704, communications unit/interface 706, a memory unit 708 in which the one or more processor unit(s) 702 are connected to the input/output unit 704, communications unit/interface 706, and the memory unit 708. In some embodiments, the computing apparatus/system 700 may be a server, or one or more servers networked together. In some embodiments, the computing apparatus/system 700 may be a computer or supercomputer/processing facility or hardware/software suitable for processing or performing the one or more aspects of the system(s), apparatus, method(s), and/or process(es) combinations thereof, modifications thereof, and/or as described herein. The communications interface 706 may connect the computing apparatus/system 700, via a communication network, with one or more services, devices, the server system(s), cloud-based platforms, systems for implementing subject-matter databases for implementing the invention as described herein. The memory unit 708 may store one or more program instructions, code or components such as, by way of example only but not limited to, an operating system and/or code/component(s) associated with the process(es)/method(s) as described with reference to the previous figures, additional data, applications, application firmware/software and/or further program instructions, code and/or components associated with implementing the functionality and/or one or more function(s) or functionality associated with one or more of the method(s) and/or process(es) of the device, service and/or server(s) hosting the process(es)/method(s)/system(s), apparatus, mechanisms and/or system(s)/platforms/architectures for implementing the invention as described herein, combinations thereof, modifications thereof, and/or as described with reference to at least one of the previous figures.

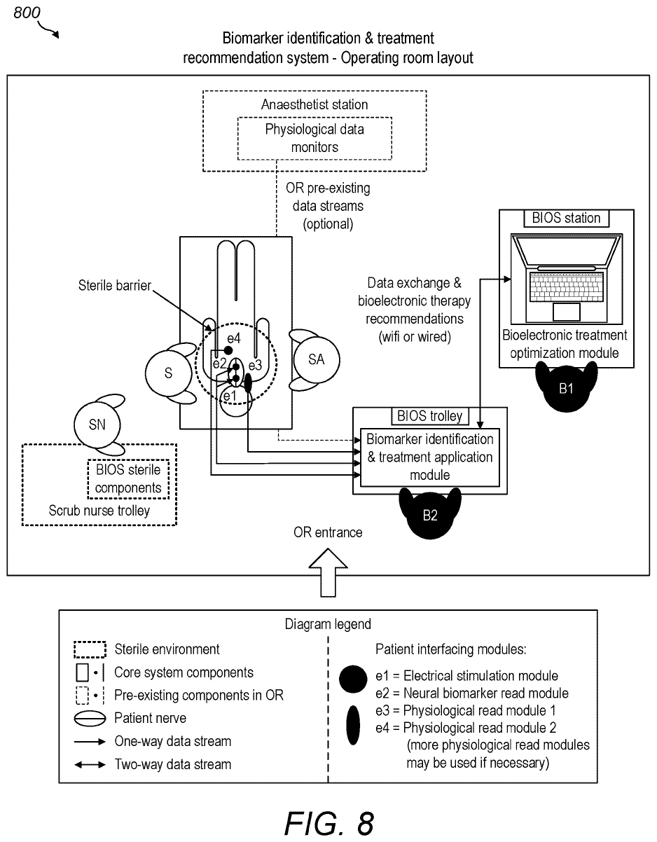

FIG. 8 is a detailed pictorial diagram illustrating an example of a clinical use environment (in surgery) for the present invention according to at least one aspect described herein. In the figure, the system is set up in relation to other BIOS devices according to FIGS. 6 and 7. The system is set up within the environment, an operating room of a physician “S” (and the physician assistants “SN” and “SA”) performing surgery on a patient attached to the BOIS devices. In the depicted operating room environment, various objects are labelled alongside devices for deploying the BIOS system. This system may include a clinical recommendation system or any other system described herein that aids or operates within a clinical setting. The BIOS system may also include subsystem for optimizing treatments using neural biomarkers, refining bioelectronic therapies for illnesses, or suggesting pharmacological treatments based on neural biomarkers—all designed to function effectively within a clinical environment.

In the embodiments, examples, and aspects of the invention as described above such as process(es), method(s), system(s) may be implemented on and/or comprise one or more cloud platforms, one or more server(s) or computing system(s) or device(s) according to FIG. 6. A server may comprise a single server or network of servers, the cloud platform may include a plurality of servers or network of servers. In some examples the functionality of the server and/or cloud platform may be provided by a network of servers distributed across a geographical area, such as a worldwide distributed network of servers, and a user may be connected to an appropriate one of the network of servers based upon a user location and the like.

The above description discusses embodiments of the invention with reference to a single user for clarity. It will be understood that in practice the system may be shared by a plurality of users, and possibly by a very large number of users simultaneously.

The embodiments described above may be configured to be semi-automatic and/or are configured to be fully automatic. In some examples a user or operator of the system(s)/process(es)/method(s) may manually instruct some steps of the process(es)/method(es) to be carried out.

The described embodiments of the invention a system, process(es), method(s) and the like according to the invention and/or as herein described may be implemented as any form of a computing and/or electronic device. Such a device may comprise one or more processors which may be microprocessors, controllers or any other suitable type of processors for processing computer executable instructions to control the operation of the device in order to gather and record routing information. In some examples, for example where a system on a chip architecture is used, the processors may include one or more fixed function blocks (also referred to as accelerators) which implement a part of the process/method in hardware (rather than software or firmware). Platform software comprising an operating system or any other suitable platform software may be provided at the computing-based device to enable application software to be executed on the device.

Various functions described herein can be implemented in hardware, software, or any combination thereof. If implemented in software, the functions can be stored on or transmitted over as one or more instructions or code on a computer-readable medium or non-transitory computer-readable medium. Computer-readable media may include, for example, computer-readable storage media. Computer-readable storage media may include volatile or non-volatile, removable or non-removable media implemented in any method or technology for storage of information such as computer-readable instructions, data structures, program modules or other data. A computer-readable storage media can be any available storage media that may be accessed by a computer. By way of example, and not limitation, such computer-readable storage media may comprise RAM, ROM, EEPROM, flash memory or other memory devices, CD-ROM or other optical disc storage, magnetic disc storage or other magnetic storage devices, or any other medium that can be used to carry or store desired program code in the form of instructions or data structures and that can be accessed by a computer. Disc and disk, as used herein, include compact disc (CD), laser disc, optical disc, digital versatile disc (DVD), floppy disk, and blu-ray disc (BD). Further, a propagated signal is not included within the scope of computer-readable storage media. Computer-readable media also includes communication media including any medium that facilitates transfer of a computer program from one place to another. A connection or coupling, for instance, can be a communication medium. For example, if the software is transmitted from a website, server, or other remote source using a coaxial cable, fiber optic cable, twisted pair, DSL, or wireless technologies such as infrared, radio, and microwave are included in the definition of communication medium. Combinations of the above should also be included within the scope of computer-readable media.

Alternatively, or in addition, the functionality described herein can be performed, at least in part, by one or more hardware logic components. For example, and without limitation, hardware logic components that can be used may include Field-programmable Gate Arrays (FPGAs), Program-specific Integrated Circuits (ASICs), Program-specific Standard Products (ASSPs), System-on-a-chip systems (SOCs). Complex Programmable Logic Devices (CPLDs), etc.

Although illustrated as a single system, it is to be understood that the computing device may be a distributed system. Thus, for instance, several devices may be in communication by way of a network connection and may collectively perform tasks described as being performed by the computing device.

Although illustrated as a local device it will be appreciated that the computing device may be located remotely and accessed via a network or other communication link (for example using a communication interface).

The term ‘computer’ is used herein to refer to any device with processing capability such that it can execute instructions. Those skilled in the art will realise that such processing capabilities are incorporated into many different devices and therefore the term ‘computer’ includes PCs, servers, IoT devices, mobile telephones, personal digital assistants and many other devices.

Those skilled in the art will realise that storage devices utilised to store program instructions can be distributed across a network. For example, a remote computer may store an example of the process described as software. A local or terminal computer may access the remote computer and download a part or all of the software to run the program. Alternatively, the local computer may download pieces of the software as needed, or execute some software instructions at the local terminal and some at the remote computer (or computer network). Those skilled in the art will also realise that by utilising conventional techniques known to those skilled in the art that all, or a portion of the software instructions may be carried out by a dedicated circuit, such as a DSP, programmable logic array, or the like.

It will be understood that the benefits and advantages described above may relate to one embodiment or may relate to several embodiments. The embodiments are not limited to those that solve any or all of the stated problems or those that have any or all of the stated benefits and advantages. Variants should be considered to be included into the scope of the invention.

Any reference to ‘an’ item refers to one or more of those items. The term ‘comprising’ is used herein to mean including the method steps or elements identified, but that such steps or elements do not comprise an exclusive list and a method or apparatus may contain additional steps or elements.

As used herein, the terms “component” and “system” are intended to encompass computer-readable data storage that is configured with computer-executable instructions that cause certain functionality to be performed when executed by a processor. The computer-executable instructions may include a routine, a function, or the like. It is also to be understood that a component or system may be localized on a single device or distributed across several devices. Further, as used herein, the term “exemplary”, “example” or “embodiment” is intended to mean “serving as an illustration or example of something”. Further, to the extent that the term “includes” is used in either the detailed description or the claims, such term is intended to be inclusive in a manner similar to the term “comprising” as “comprising” is interpreted when employed as a transitional word in a claim.

The figures illustrate exemplary methods. While the methods are shown and described as being a series of acts that are performed in a particular sequence, it is to be understood and appreciated that the methods are not limited by the order of the sequence. For example, some acts can occur in a different order than what is described herein. In addition, an act can occur concurrently with another act. Further, in some instances, not all acts may be required to implement a method described herein.

Moreover, the acts described herein may comprise computer-executable instructions that can be implemented by one or more processors and/or stored on a computer-readable medium or media. The computer-executable instructions can include routines, sub-routines, programs, threads of execution, and/or the like. Still further, results of acts of the methods can be stored in a computer-readable medium, displayed on a display device, and/or the like.

The order of the steps of the methods described herein is exemplary, but the steps may be carried out in any suitable order, or simultaneously where appropriate. Additionally, steps may be added or substituted in, or individual steps may be deleted from any of the methods without departing from the scope of the subject matter described herein. Aspects of any of the examples described above may be combined with aspects of any of the other examples described to form further examples without losing the effect sought.

It will be understood that the above description of a preferred embodiment is given by way of example only and that various modifications may be made by those skilled in the art.

What has been described above includes examples of one or more embodiments. It is, of course, not possible to describe every conceivable modification and alteration of the above devices or methods for purposes of describing the aforementioned aspects, but one of ordinary skill in the art can recognize that many further modifications and permutations of various aspects are possible. Accordingly, the described aspects are intended to embrace all such alterations, modifications, and variations that fall within the scope of the appended claims.

1. A clinical recommendation system, wherein the system comprising: one or more modules configured to:
obtain a set of neural signal data collected from the patient suffering from the illness, wherein the set of neural signal data is collected with respect to a neural biomarker;
obtain an assessment based on the set of neural signal data in relation to a response of a neural biomarker from the patient, wherein the biomarker response is indicative of one or more determinations made with respect to the neural biomarker by the system; and
provide the assessment, at least in part, as a recommendation that corresponds to the patient.

2. The system of claim 1, wherein the system is configured to provide a recommendation concerning one or more treatments for an illness based on the neural biomarker response.

3. The system of claim 1 or 2, wherein at least one module is configured to:
receive the set of neural signal data;
determine a baseline for the neural biomarker response from the set of neural signal data in the absence of the treatment;
administer a treatment based on a schedule to the patient while obtaining the set of neural signal data collected from the patient;
identify one or more changes in the set of neural signal data in response to and for a duration of the treatment;
determine the neural biomarker response based on said one or more changes in the set of neural signal data through the time period appropriate to the treatment;
ascertain the neural biomarker response in relation to the baseline;
provide an assessment of the treatment based on the neural biomarker response; and
output the assessment as the recommendation.

4. The system of claims 1 or 2, wherein at least one module is configured to:
receive the set of neural signal data;
determine a level of the neural biomarker present with respect to a historical patient population with the illness and/or patient specific levels of the neural biomarker associated with the illness;
determine a state or severity of the illness based on one or more determined levels of the neural biomarker as compared to the historical patient population or the patient specific levels of neural biomarker characteristics associated with the illness;
provide an assessment of one or more treatments based on said determinations, wherein the assessment correlates one or more neural biomarker levels in relation to those of, or known for, the illness; and
output the assessment as the recommendation.

5. The system of claim 1 or 2, wherein at least one module is configured to:
receive the set of neural signal data;
determine, in the absence of treatment for the illness, the neural biomarker response for an expected response of a treatment based on a patient population with the treatment and/or historical levels of the neural biomarker response from a different patient in response to the treatment;
provide an assessment of one or more treatments based on the determination in relation to the neural biomarker response historically known for the illness; and
output the assessment as the recommendation.

6. The system of any preceding claims, wherein the assessment comprises one or more proposed modifications to a schedule of the treatment.

7. The system of any preceding claims, wherein the set of neural signal data is collected by an external device in a continuous and uninterrupted manner.

8. The system of any preceding claims, wherein the set of neural signal data is collected and stored on an external device.

9. The system of any preceding claims, wherein the set of neural signal data is collected for a time interval.

10. The system of claim 9, wherein the time interval begins from a first time point before a treatment is administered and ends at a second time point after the treatment has been administered.

11. The system of any preceding claims, wherein the set of neural signal data is obtained in real-time as a treatment is being administered.

12. The system of any preceding claim, wherein the set of neural signal data is obtained from one of:
an implantable neurally-connected device with continuously recording functionality; an implantable neurally-connected device that records neural data either in batches or only when powered or wirelessly connected to a second external device; a neurally-connected device that temporarily goes through the skin to a nerve for the duration of recording; a neurally-connected device that is temporarily situated inside a patient and can be connected to by a second device for periods of recording; a fully non-invasive neural device that detects neural activity wirelessly at a distance from the nerve; a system that detects neural activity based on a prior procedure to allow detection; a device with a system that is configured to detect non-neural signals that themselves contain proxies of neural data; or a device employing one or more methods of detecting neural data or proxies of neural data.

13. The system of any preceding claims, wherein the set of neural signal data comprises time series data capturing different treatment stages in relation to one or more physiological responses exhibited by the patient during the different treatment stages.

14. The system of claims 2 to 13, wherein each treatment is selected from a set of available treatments based on a physiological condition of the patient in relation to a determined progression of the illness.

15. The system of claims 2 to 14, wherein each treatment is further selected from a set of available treatments based on the neural biomarker response of the patient in relation to the treatment and/or the illness.

16. The system of any preceding claims, further comprising:
a set of treatments associated with the illness, wherein each treatment of the set is trialled with a patient and a preferred treatment is selected based on the neural biomarker response of the patient in relation to the set of treatment and/or the illness.

17. The system of claims 2 to 16, wherein said one or more treatments comprise bioelectronic therapies and/or molecular therapies.

18. The system of claim 17, wherein the bioelectronic therapies comprise an electrical stimulation of a nerve of the patient.

19. The system of claim 17 or 18, wherein the treatment is a bioelectronic treatment and the neural biomarker response is between 0.5 ms to 5 ms following the treatment.

20. The system of claims 17 to 19, wherein the treatment is a bioelectronic treatment and the neural biomarker response is between 5 s to 60 s following the treatment.

21. The system of claims 17 or 20, wherein the molecular therapies comprise an administration of a medicament to the patient.

22. The system of claims 17 or 21, wherein the treatment is a molecular treatment and the neural biomarker response is between 1 s to 180 s following the treatment.

23. The system of claims 17 or 22, wherein the treatment is a molecular treatment and the neural biomarker response is between 0.5 hr to 4 hr and/or 1 hr to 24 hr following the treatment.

24. The system of claim 23, wherein the illness is a respiratory inflammatory illness.

25. The system of claims 3 and 6 to 24, wherein said one or more changes exhibited in the set of neural signal data are statistically significant in relation to the set of neural signal data at a time point before the treatment is administered.

26. The system of claims 3 and 6 to 24, wherein said one or more changes correspond to at least one change or improvement in one or more physiological parameters of the patient with respect to the treatment.

27. The system of any preceding claims, wherein the neural biomarker response is induced by the onset, or progression, of the illness and/or the treatment.

28. The system of any preceding claims, wherein the illness comprises a neurally-mediated and/or neural-related disease and/or condition.

29. The system of any preceding claims, wherein the assessment comprises a schedule of a treatment that follows a pre-determined threshold dosage that is set based on historical treatment data associated with illness.

30. The system of any preceding claims, wherein said one or more modules are configured to:
measure the neural biomarker response at a time point following treatment;
adjust a treatment based on whether the measured response at the time point meets a pre-determined neural biomarker response threshold;
modify a schedule of the treatment in relation to the adjustment, wherein the schedule is adapted to include the adjustment based on a pre-determined treatment plan; and
output the schedule of the treatment.

31. The system of any preceding claims, wherein said one or more modules are configured to:
determine one or more physiological parameters of the patient prior to initiating the treatment;
identify a treatment based on said one or more physiological parameters;
initiate the treatment to elicit the neural biomarker response; and
adapt the treatment based on a change in at least one physiological parameter in relation to the neural biomarker response.

32. The system of any preceding claims, wherein said one or more modules are configured to:
measure the neural biomarker response of a treatment for a time interval; and
update a pre-determined neural biomarker response threshold based on the measurement.

33. The system of claim of any preceding claims, further comprising: at least one module that is configured to: provide one or more types of electrical stimulation to a nerve of the patient exhibiting the treatment.

34. The system of any preceding claims, further comprising: at least one module that is configured to: induce the treatment based on a change in a physiological parameter of the patient in relation to the illness.

35. The system of any preceding claim, wherein the neural biomarker is used to guide dosing of a treatment.

36. A method for providing an assessment based on a neural biomarker response, the method comprising:
obtaining a set of neural signal data collected from the patient suffering from the illness, wherein the set of neural signal data is collected with respect to a neural biomarker;
obtaining an assessment based on the set of neural signal data in relation to a response of a neural biomarker from the patient, wherein the biomarker response is indicative of one or more determinations made with respect to the neural biomarker by the system; and
providing the assessment for the patient.

37. The method of claim 36, wherein the method is used in conjunction with or as part of the system according to any of claims 2 to 35, wherein the system comprising: an external device for obtaining the set of neural signal data used by the method.

38. A device for implementing the system according to claims 1 to 35, wherein the device comprising: at least one component configured to: identify a neural biomarker for use with the system based on the method of claims 39 to 50.

39. A method for identifying a neural biomarker based on neural signal data associated with an illness, the method comprising:
receiving a set of neural signal data, wherein the set of neural signal data is collected from a patient during a progression of the illness;
identifying a pattern of change in the set of neural signal data;
determining the neural biomarker by associating the pattern of change with said progression of the illness; and
providing the neural biomarker for use in one or more medical applications in relation to the illness.

40. The method of claim 39, further comprising:
administering a treatment for the illness to the patient;
receiving a set of neural signal data collected from a patient a period following the treatment;
comparing the pattern of change following the treatment with that in the absence of the treatment;
identifying a change with respect to the treatment in the pattern of change in the set of neural signal data; and
ascertaining the neural biomarker based on the identified change.

41. The method of claim 39 or 40, wherein the pattern of change is associated with at least one deviation of: signal data collected from the patient, historical neural signal data of the patient, historical neural signal data associated with a population of patients, a historical neural signal data of a non-human subject with the illness and/or being administered the treatment.

42. The method of claims 39 to 41, further comprising:
receiving a set of physiological data corresponding to the set of neural signal data;
identifying the initial pattern of change in the set of neural signal data based on historical neural signal data obtained from a population without the illness; and
confirming the neural biomarker based on the pattern of change in relation to the set of physiological data, wherein the set of physiological data is at least partially indicative of said progression of the illness without treatment.

43. The method of claims 39 to 42, further comprising:
selecting a treatment for the illness;
measuring a response to the identified neural biomarker based on the treatment with respect to the pattern of change identified; and
compiling the response as part of neural biomarker responses associated with the illness.

44. The method of claims 39 to 43, further comprising:
obtaining one or more responses for the identified neural biomarker;
compiling said one or more responses as a reference set, wherein the reference set is structured with respect to or to reflect said progression of the illness for the patient, for a sub-population of patients associated with the patient, or for a cohort of patients suffering from the illness;
providing a personalised neural biomarker dose response based on the reference set; and
updating a treatment plan for that patient based on the personalised neural biomarker dose response, wherein the treatment plan comprises at least a dosage plan.

45. The method of claims 39 to 44, further comprising:
selecting the treatment in relation to said one or more medical applications; and
adapting the treatment based on the neural biomarker responses.

46. The method of claims 39 to 45, further comprising:
performing said one or more medical applications using the neural biomarker; and
adapting said one or more medical applications in accordance with a response from the neural biomarker.

47. The method of claims 39 to 46, further comprising:
obtaining one or more responses for the identified neural biomarker;
compiling said one or more responses as a set of historical neural biomarker responses that is used as the baseline in the system according to any of claims 1 to 23 for the patient or a population of patients with the illness that has been administered with that treatment; and
providing the assessment from said system with respect to and in consideration of the set of historical neural biomarker responses.

48: The method of claims 39 to 47, further comprising:
providing the neural biomarker to be used with an algorithm; and
allowing the algorithm to update and/or adapt the treatment or a treatment plan towards a target therapeutic objective,
wherein the target therapeutic objective is defined on the basis of a target neural biomarker, a target physiological response, a clinician-defined observation, and/or a combination thereof,
wherein the algorithm is configured to optimize for the target therapeutic objective based on the set of neural signal data.

49. The method of claim 48, wherein the algorithm is configured to: optimize periodically with respect to days, weeks, or months as the treatment plan is being or to be updated with respect to a long-term target therapeutic objective.

50. The method of claim 48 or 49, wherein the algorithm comprises one or more Bayesian optimization techniques.

51. A system for optimizing a treatment based on a neural biomarker, wherein the system is configured to: perform the method steps according to any of the claims 48 to 50 in order to optimize the treatment or the treatment plan toward the target therapeutic objective for the illness in relation to or as part of the recommendation provided by the system according to any of claims 1 to 35.

52. A method for applying neural biomarker response in clinical diagnosis and treatment screening, the method comprising:
obtaining a neural biomarker response in response to a treatment for an illness exhibited by a patient;
determining whether the neural biomarker response meets one or more pre-determined criteria determined based on a patient cohort exhibiting neural biomarker response following successful treatment(s) or recovery of the illness; and
providing the treatment based on whether neural biomarker response meets said one or more pre-determining criteria, wherein the treatment is used in relation to the clinical diagnosis and treatment screening.

53. The method of claim 52, wherein the biomarker response is obtained for a neural biomarker identified according to any of the methods 39 to 50.

54. The method of claim 52 or 53, wherein said one or more pre-determined criteria are associated with at least one physiological parameter of the patient.

55. The method of claims 52 to 54, wherein clinical diagnosis and treatment screening comprise one or more stages of the clinical trials.

56. The method of claims 52 to 55, further comprising:
generating a schedule of the treatment based on the neural biomarker response, wherein the schedule is formulated in relation to one or more physiological parameters of the patient following the treatment.

57. A system for optimizing a bioelectronic treatment for an illness, the system comprising one or more modules configured to:
apply an electrical stimulation to a nerve of the patient based on a set of electrical stimulation parameters;
obtain a set of neural signal data from the patient in relation to the electrical stimulation;
determine one or more neural biomarker responses from changes in:
the set of neural signal data indicative of changes in neural activity, wherein the changes are associated with the progression of the illness, in response to the bioelectronic treatment, or when undergoing the bioelectronic treatment, or for a period following the bioelectronic treatment, or a combination thereof;
obtain one or more physiological signals of the patient in response to the bioelectronic treatment; and
transmit said one or more neural biomarker responses and/or one or more physiological signals to be analysed by an optimizer, wherein the optimizer is configured to:
receive said one or more neural biomarker responses and/or said one or more physiological signals as input data to a model or search algorithm related to the model;
evaluate the input data based an objective, wherein the objective comprise a set of recommendation or a predetermined set of rules;
determine a query point based on the objective; and
output the query point;
updated the set of electrical stimulation parameters based on one or more outputs from the optimizer.

58. The system of claim 57, wherein said one or more neural biomarker responses comprise at least one response of the neural fibre types to electrical stimulation of the nerve.

59. The system of claim 57 or 58, wherein said one or more neural biomarker responses comprise at least one response of neural fibres within different spatial locations of the nerve.

60. The system of claims 57 to 59, wherein said one or more neural biomarker responses comprise measurements of a neurally-mediated process.

61. The system of claims 57 to 60, wherein the updated set of electrical stimulation parameters is delivered automatically by a device configured to implement the model.

62. The system of claims 57 to 61, wherein the system for optimizing the bioelectronic treatment would run through multiple iterations and the electrical stimulation parameters are updated for each iteration.

63. The system of claims 57 to 62, wherein the updated set of electrical stimulation parameters is reviewed and/or modified by a clinician.

64. The system of claims 57 to 63, wherein the updated set of electrical stimulation parameters is processed by a second model to assess for safety.

65. The system of claims 57 to 64, wherein the set of electrical stimulation parameters is associated with square wave stimulations comprising one or more of: a current, a pulse width, a train frequency, a polarity, a train duration, a duty cycle on/off time, a spatial location.

66. The system of claims 57 to 65, wherein the set of neural signal data is controlled for neural data quality, and the optimization takes into account the neural data quality during each update.

67. The system of claims 57 to 66, furthering comprising:
optimizing the bioelectronic treatment using Bayesian optimization based on one or more electrical stimulation parameters, wherein the bioelectronic treatment comprises at least one vagus nerve stimulation.

68. The system of claims 57 to 67, wherein the updated set of electrical stimulation parameters is used for training at least one model configured to:
identify one or more changes in a set of neural signal data indicative of a neural biomarker response; and
ascertain the neural biomarker response based on said one or more changes for the duration of the treatment.

69. The system of claim 68, further comprising:
applying the updated set of electrical stimulation parameters to obtain a set of neural signal data collected from the patient before, during, and after the treatment.

70. The system of claim 69, further comprising:
applying the set of neural signal data to the system of any of claims 1 to 35 to obtain a recommendation based on the updated set of electrical stimulation parameters.

71. A system for recommending a pharmacological treatment based on a neural biomarker, the system comprising one or more modules configured to:
receive a set of neural signal data of the patient;
determine a neural biomarker response from one or more changes in the set of neural signal data indicative of neural activity before, during, and after the treatment with a medicament; and
provide recommendations on whether a threshold dosage has been reached in relation to the neural biomarker response and/or provide recommendations on whether the patient is responding to the treatment on the basis of the neural biomarker response.

72. The system of claim 71, wherein the neural biomarker is identified according to the method of any of the claims 39 to 50.

73. A device for performing optimization based on an identified neural biomarker according to a method of any of claims 39 to 50 and/or to implement the optimization steps according to the system of any of claims 57 to 69.

74. The system of any preceding claims, wherein system is further configured have at least one module for providing a treatment recommendation, where the treatment recommendation comprises a treatment different to a treatment being assessed by the system, and/or data corresponding to behavioural changes of the patient.

75. The system of claim 74, wherein said different treatment comprises a bioelectronic treatment, a surgical ablation/denervation, and a treatment by administering one or more compounds.

76. An apparatus for controlling one or more devices configured to provide a health-related recommendation for a patient, wherein the apparatus is configured to instruct at least one device to:
identify a neural biomarker according to a method of any of claims 39 to 50;
retrieve a set of neural signal data from the patient; determine a neural biomarker response for the identified neural biomarker based on the set of neural signal data; and
provide a clinical recommendation based on the neural biomarker response according to a system any of claims 1 to 35, and/or
optimize a treatment based on the identified neural biomarker or for an illness according to a system any of claims 57 to 70, and/or
recommend a pharmacological treatment based on a neural biomarker according to a system of claim 70 or 71.

77. The system or method of any preceding claims, wherein the neural biomarker response is determined by using one or more machine learning techniques based on the set of neural signal data.

78. The system or method of claim 77, wherein said one or more machine learning techniques are configured to combine the set of neural signal data with other data collected in relation to the patient.

79. The system or method of any preceding claims, wherein the set of neural signal data or the input data during optimization is processed using said one or more machine learning techniques.

80. The method of any preceding claims, further composing:
applying one or more machine learning models to an input or output the system according to any preceding claims, wherein said one or more machine learning models are trained with respect to a neural biomarker response using the set of neural signal data, data associated with a historical patient population with an illness, and/or data of patient specific levels of the neural biomarker associated with an illness.
